(19) United States
(12) Patent Application Publication
(10) Pub. No.: US 2016/0166326 A1
(43) Pub. Date: Jun. 16, 2016

(54) MEDICAL LEAD BENDING SENSOR

(71) Applicant: Medtronic Bakken Research Center B.V., Maastricht (NL)

(72) Inventors: Egbertus Johannes Maria Bakker, Wijk en aalburg (NL); Jeroen Jacob Arnold Tol, Eindhoven (NL); Sebastien Jody Ouchouche, Waalre (NL)

(21) Appl. No.: 14/952,672

(22) Filed: Nov. 25, 2015

Related U.S. Application Data
(60) Provisional application No. 62/084,312, filed on Nov. 25, 2014.

(51) Int. Cl. A61B 19/00; A61N 1/05; A61B 5/0492

(52) U.S. Cl. CPC A61B 19/46; A61N 1/0534; A61B 5/0492; A61B 2019/464

(57) ABSTRACT

In some example, a medical device system including a medical lead; a bending sensor; and a controller configured to sense a bending of the medical lead during implantation of the medical lead in a patient based on the output of the bending sensor. The systems and techniques of this disclosure may improve the accuracy of the implantation of neurostimulation medical leads, for example, by accounting for bending deformation of the medical lead during implantation.

This application claims the benefit of U.S. Provisional Application No. 62/084,312, filed Nov. 25, 2014, the entire content of which is incorporated by reference herein.

### TECHNICAL FIELD

This disclosure relates, in some examples, to medical leads and medical device systems.

### BACKGROUND

Implantable neurostimulation devices may treat acute or chronic neurological conditions. Deep brain stimulation (DBS), which may include, e.g., the mild electrical stimulation of cortical and/or sub-cortical structures, belongs to this category of implantable devices, and has been shown to be therapeutically effective for such conditions as Parkinson's disease, Dystonia, Epilepsy, Alzheimer's Disease, and Tremor. As another example, DBS may be used to treat psychiatric disorders (obsessive-compulsive disorder, depression). DBS systems may include one or more leads connected to an implantable pulse generator.

### SUMMARY

In some examples, the disclosure relates to systems and techniques for monitoring bending of a medical lead during implantation of the medical lead, e.g., in a tissue of a patient. The described systems and techniques may facilitate precise positioning of the medical lead within a tissue of the patient, such as the brain of the patient. Precise positioning of the medical lead may allow better targeting of tissues within a patient, whether for stimulation, sensing or both.

In one example, this disclosure is directed to a medical device system comprising a medical lead; a bending sensor; and a controller configured to sense a bending of the medical lead during implantation of the medical lead in a patient based on the output of the bending sensor.

In another example, this disclosure is directed to a method for guiding implantation of a medical lead, the method comprising while the medical lead is being inserted into tissue of a patient, monitoring a signal from a bending sensor associated with the medical lead; evaluating bending of the medical lead during the insertion based on the monitored signal; and while the medical lead is being inserted into the tissue of the patient, generating information to guide steering of the medical lead toward a target site based on the evaluation of the bending of the medical lead, wherein at least one of the monitoring, evaluating, or generating is performed via a processor.

In a further example, this disclosure is directed to a system comprising a medical lead; means for monitoring a signal from a bending sensor associated with the medical lead while the medical lead is being inserted into tissue of a patient; means for evaluating bending of the medical lead during the insertion based on the monitored signal; and means for generating information, while the medical lead is being inserted into the tissue of the patient, to guide steering of the medical lead toward a target site based on the evaluation of the bending of the medical lead.

The details of one or more examples of this disclosure may be set forth in the accompanying drawings and the description below. Other features, objects, and advantages of this disclosure may be apparent from the description and drawings, and from the claims.

### BRIEF DESCRIPTION OF DRAWINGS

FIG. 1 is a conceptual diagram illustrating an example deep brain stimulation (DBS) system configured to sense a bioelectrical brain signal and deliver electrical stimulation therapy to a tissue site within a brain of a patient.

FIG. 2 is functional block diagram illustrating components of an example medical device including a stimulation generator.

FIG. 3 is a functional block diagram illustrating components of an example medical device system including an implantable pulse generator and a separate active medical lead can with a switch matrix to direct signals from the implantable pulse generator to different electrodes.

FIG. 4 is a functional block diagram illustrating components of another example medical device system including an implantable pulse generator and a separate active medical lead can with a switch matrix to direct signals from the implantable pulse generator to different electrodes.

FIGS. 5A-5C illustrate examples of medical leads for stimulation and/or sensing that may be used in the systems of FIGS. 1, 3 and 4.

FIG. 6 illustrates a medical device system including a medical lead and a stylet that includes an optical bending sensor.

FIG. 7 illustrates a medical lead that includes an optical bending sensor.

FIG. 8 illustrates a medical device system including a medical lead and a stylet that includes a bending sensor with a piezoelectric sensor.

FIG. 9 illustrates a medical lead that includes a bending sensor with a piezoelectric sensor.

FIG. 10 illustrates a medical device system including a medical lead and a stylet that includes a bending sensor with a resistance sensor.

FIG. 11 illustrates a medical lead that includes a bending sensor with a resistance sensor.

FIG. 12 is a functional block diagram illustrating components of an example system including a medical lead, a bending sensor and a controller that evaluates bending of the medical lead.

FIG. 13 is a functional block diagram illustrating components of an example medical device programmer.

FIG. 14 is a flowchart illustrating an example technique for guiding implantation a medical lead including monitoring a signal from a bending sensor associated with the medical lead while the medical lead is being inserted into tissue of a patient.

FIG. 15 is a flowchart illustrating an example technique for controlling therapy or sensing with a medical lead based on a monitored a signal from a bending sensor associated with the medical lead.

### DETAILED DESCRIPTION

The accurate positioning of one or more electrodes carried by an implantable neurostimulation medical lead relative to target tissue or nerve structures can be very important. Accordingly, during implantation of a medical lead, any help to improve the accuracy by guiding implantation is welcome. The systems and techniques of this disclosure may, in some examples, improve the accuracy of the implantation of neurostimulation medical leads. For example, a system for neurostimulation and/or neurorecording may include at least one medical lead and at least one bending sensor, the bending sensor being configured to sense a bending of the medical lead during the implantation. In some examples, the system may output a signal indicative of the bending to facilitate more precise positioning of the medical lead during implantation.

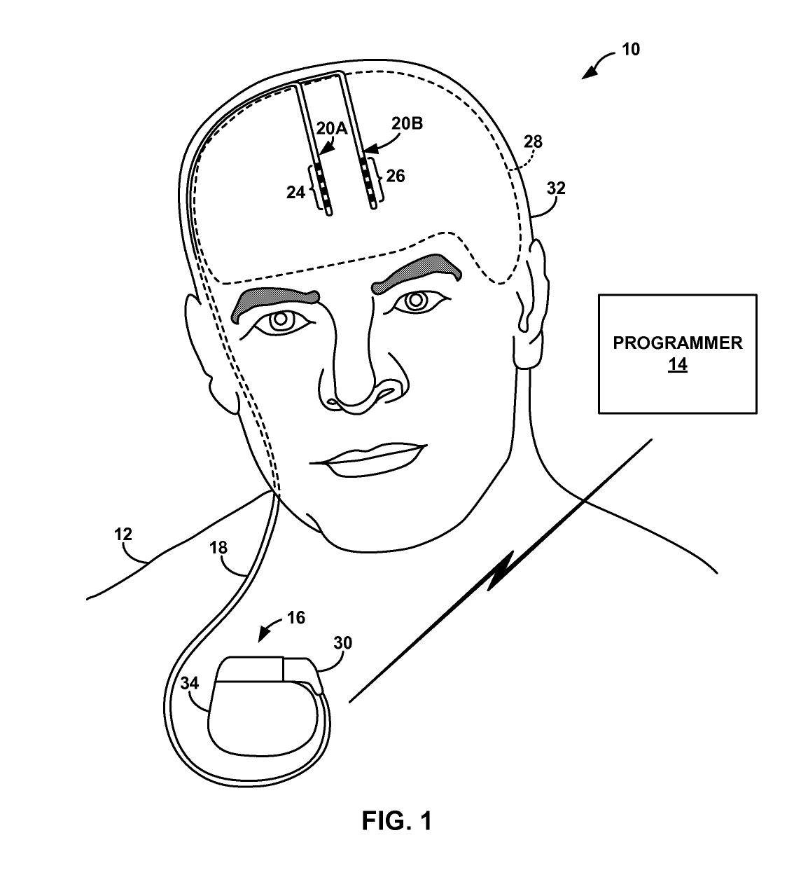

FIG. 1 is a conceptual diagram illustrating an example therapy system 10 that is configured to deliver therapy to patient 12 to manage a disorder of patient 12. Patient 12 ordinarily will be a human patient. In some cases, however, therapy system 10 may be applied to other mammalian or non-mammalian non-human patients. In the example shown in FIG. 1, therapy system 10 includes medical device programmer 14, implantable medical device (IMD) 16, medical lead extension 18, and one or more medical leads 20A and 20B (collectively “medical leads 20”) with respective sets of electrodes 24, 26. IMD 16 includes a stimulation generator configured to generate and deliver electrical stimulation therapy to one or more regions of brain 28 of patient 12 via one or more electrodes 24, 26 of medical leads 20A and 20B, respectively, alone or in combination with an electrode provided by outer housing 34 of IMD 16.

In the example shown in FIG. 1, therapy system 10 may be referred to as a DBS system because IMD 16 is configured to deliver electrical stimulation therapy directly to tissue within brain 28, e.g., a tissue site under the dura mater of brain 28 or one or more branches or nodes, or a confluence of fiber tracks. In other examples, medical leads 20 may be positioned to deliver therapy to a surface of brain 28 (e.g., the cortical surface of brain 28). For example, in some examples, IMD 16 may provide cortical stimulation therapy to patient 12, e.g., by delivering electrical stimulation to one or more tissue sites in the cortex of brain 28. Frequency bands of therapeutic interest in cortical stimulation therapy may include the theta band, and the gamma band.

DBS may be used to treat or manage various patient conditions, such as, but not limited to, seizure disorders (e.g., epilepsy), pain, migraine headaches, psychiatric disorders (e.g., major depressive disorder (MDD), bipolar disorder, anxiety disorders, post-traumatic stress disorder, dysthymic disorder, and obsessive-compulsive disorder (OCD), behavior disorders, mood disorders, memory disorders, mentation disorders, movement disorders (e.g., essential tremor or Parkinson's disease), Huntington's disease, Alzheimer's disease, or other neurological or psychiatric disorders and impairment of patient 12. Therapy systems configured for treatment of other patient conditions via delivery of therapy to brain 28 can also be used in accordance with the techniques for determining one or more therapeutic windows disclosed herein.

In the example shown in FIG. 1, IMD 16 may be implanted within a subcutaneous pocket in the pectoral region of patient 12. In other examples, IMD 16 may be implanted within other regions of patient 12, such as a subcutaneous pocket in the abdomen or buttocks of patient 12 or proximate to the cranium of patient 12. Implanted medical lead extension 18 is coupled to IMD 16 via connector block 30 (also referred to as a header), which may include, for example, electrical contacts that electrically couple to respective electrical contacts on medical lead extension 18. The electrical contacts electrically couple the electrodes 24, 26 carried by medical leads 20 to IMD 16. Medical lead extension 18 traverses from the implant site of IMD 16, along the neck of patient 12 and through the cranium of patient 12 to access brain 28. IMD 16 can be constructed of a biocompatible material that resists corrosion and degradation from bodily fluids. IMD 16 may comprise a hermetic outer housing 34 to substantially enclose components, such as a processor, a therapy module, and memory.

In the example shown in FIG. 1, medical leads 20 are implanted within the right and left hemispheres, respectively, of brain 28 in order to deliver electrical stimulation to one or more regions of brain 28, which may be selected based on many factors, such as the type of patient condition for which therapy system 10 is implemented to manage. Other implant sites for medical leads 20 and IMD 16 are contemplated. For example, IMD 16 may be implanted on or within cranium 32 or medical leads 20 may be implanted within the same hemisphere at multiple target tissue sites or IMD 16 may be coupled to a single medical lead that is implanted in one or both hemispheres of brain 28.

During implantation of medical lead 16 within patient 12, a clinician may attempt to position electrodes 24, 26 of medical leads 20 such that electrodes 24, 26 are able to deliver electrical stimulation to one or more target tissue sites within brain 28 to manage patient symptoms associated with a disorder of patient 12. Medical leads 20 may be implanted to position electrodes 24, 26 at desired locations of brain 28 via any suitable technique, such as through respective burr holes in the skull of patient 12 or through a common burr hole in the cranium 32. Medical leads 20 may be placed at any location within brain 28 such that electrodes 24, 26 are capable of providing electrical stimulation to target therapy delivery sites within brain 28 during treatment and/or sense electrical activity of the patient. As described herein, implantation may include, while inserting the medical lead into the brain of patient 12, monitoring a signal from a bending sensor associated with a medical lead to evaluate bending of the medical lead during the insertion. During a DBS implantation procedure, the medical lead bends during implantation. This can cause the medical lead to deviate from its planned trajectory, while moving towards the intended target. As this deviation is hard to detect, the bending sensor may facilitate more precise positioning of the medical lead.

The anatomical region within patient 12 that serves as the target tissue site for stimulation delivered by IMD 14 may be selected based on the patient condition. Different neurological or psychiatric disorders may be associated with activity in one or more of regions of brain 28, which may differ between patients. Accordingly, the target therapy delivery site for electrical stimulation therapy delivered by medical leads 20 may be selected based on the patient condition. For example, a suitable target therapy delivery site within brain 28 for controlling a movement disorder of patient 12 may include one or more of the pedunculopontine nucleus (PPN), thalamus, basal ganglia structures (e.g., globus pallidus, substantia nigra or subthalamic nucleus), zona inserta, fiber tracts, lenticular fasciculus (and branches thereof), ansa lenticularis, or the Field of Forel (thalamic fasciculus). The PPN may also be referred to as the pedunculopontine tegmental nucleus.

As another example, in the case of MDD, bipolar disorder, OCD, or other anxiety disorders, medical leads 20 may be implanted to deliver electrical stimulation to the anterior limb of the internal capsule of brain 28, and only the ventral portion of the anterior limb of the internal capsule (also referred to as a VC/VS), the subgenual component of the cingulate cortex (which may be referred to as CG25), anterior cingulate cortex Brodmann areas 32 and 24, various parts of the prefrontal cortex, including the dorsal lateral and medial pre-frontal cortex (PFC) (e.g., Brodmann area 9), ventromedial prefrontal cortex (e.g., Brodmann area 10), the lateral and medial orbitofrontal cortex (e.g., Brodmann area 11), the medial or nucleus accumbens, thalamus, intralaminar thalamic nuclei, amygdala, hippocampus, the lateral hypothalamus, the Locus ceruleus, the dorsal raphe nucleus, ventral tegmentum, the substantia nigra, subthalamic nucleus, the inferior thalamic peduncle, the dorsal medial nucleus of the thalamus, the habenula, the bed nucleus of the stria terminalis, or any combination thereof.

As another example, in the case of a seizure disorder or Alzheimer's disease, for example, medical leads 20 may be implanted to deliver electrical stimulation to regions within the Circuit of Papez, such as, e.g., one or more of the anterior thalamic nucleus, the internal capsule, the cingulate, the fornix, the mammillary bodies, the mammillothalamic tract (mammillothalamic fasciculus), or the hippocampus. Target therapy delivery sites not located in brain 28 of patient 12 are also contemplated.

The techniques of this disclosure may be implemented in combination with systems including smaller electrodes, such as electrodes manufactured using thin film manufacturing. Examples of such manufacturing techniques for a medical lead made from a thin film based on thin film technology are disclosed in United States Patent Application Publication No. 2011/0224765, titled, “SPIRALED WIRES IN A DEEP-BRAIN STIMULATION PROBE,” the entire contents of which are incorporated by reference herein. The thin film medical leads may be fixed on a core material to form a medical lead. These medical leads may include multiple electrode areas and may enhance the precision to address the appropriate target in the brain and relax the specification of positioning. Meanwhile, undesired side effects due to undesired stimulation of neighboring areas may be limited.

Other examples of such manufacturing techniques for a medical lead based on thin film manufacturing are disclosed in U.S. Pat. No. 7,941,202, titled, “MODULAR MULTICHANNEL MICROELECTRODE ARRAY AND METHODS OF MAKING SAME,” the entire contents of which are incorporated by reference herein.

Although medical leads 20 are shown in FIG. 1 as being coupled to a common medical lead extension 18, in other examples, medical leads 20 may be coupled to IMD 16 via separate medical lead extensions or directly coupled to IMD 16. Moreover, although FIG. 1 illustrates system 10 as including two medical leads 20A and 20B coupled to IMD 16 via medical lead extension 18, in some examples, system 10 may include one medical lead or more than two medical leads.

In the examples shown in FIG. 1, electrodes 24, 26 of medical leads 20 are shown as ring electrodes that extend around the entire circumference of the lead body. Ring electrodes may be relatively easy to program and may be capable of delivering an electrical field to any tissue adjacent to medical leads 20. In other examples, electrodes 24, 26 of medical leads 20 may have different configurations. For example, one or more of the electrodes 24, 26 of medical leads 20 may have a complex electrode array geometry that is capable of producing shaped electrical fields, including interleaved stimulation.

An example of a complex electrode array geometry may include an array of electrodes positioned at different axial positions along the length of a medical lead, as well as at different angular positions about the periphery, e.g., circumference, of the medical lead. The complex electrode array geometry may include multiple electrodes (e.g., partial ring or some other segmented electrodes that may have any other shape other than a partial ring) around the perimeter of each medical lead 20, in addition to, or instead of, a ring electrode. In other examples, the complex electrode array geometry may include electrode pads distributed axially and circumferentially about the medical lead 20. In either case, these such segmented (or directional) electrodes extend only part of the way around the full circumference of the lead so that electrical stimulation may be directed to a specific direction from medical leads 20 to enhance therapy efficacy and reduce possible adverse side effects from stimulating a large volume of tissue. This is in contrast to the full ring electrodes which do extend around the full circumference of the lead body, and which provides stimulation around the entire lead circumference.

In some examples, both ring and segmented electrodes are provided by the lead. One example of such a lead includes a so-called “1-3-3-1” lead having a distal ring or distal tip electrode. Two rows of three segmented electrodes are located proximal to this distal-most electrode. A more proximal ring electrode is provided proximal to the two rows of three segmented electrodes. Such a lead is described in U.S. Pat. No. 7,668,601 assigned to the assignee of the current application and incorporated herein by reference.

In some examples, outer housing 34 of IMD 16 may include one or more stimulation and/or sensing electrodes. For example, housing 34 can comprise an electrically conductive material that is exposed to tissue of patient 12 when IMD 16 is implanted in patient 12, or an electrode can be attached to housing 34. In other examples, medical leads 20 may have shapes other than elongated cylinders as shown in FIG. 1 with active or passive tip configurations. For example, medical leads 20 may be paddle medical leads, spherical medical leads, bendable medical leads, or any other type of shape effective in treating patient 12.

IMD 16 may deliver electrical stimulation therapy to brain 28 of patient 12 according to one or more therapy programs. A therapy program may define one or more electrical stimulation parameter values for therapy generated by a stimulation generator of IMD 16 and delivered from IMD 16 to a target therapy delivery site within patient 12 via one or more electrodes 24, 26. The electrical stimulation parameters may define an aspect of the electrical stimulation therapy, and may include, for example, voltage or current amplitude of an electrical stimulation signal, a frequency of the electrical stimulation signal, and, in the case of electrical stimulation pulses, a pulse rate, a pulse width, a waveform shape, and other appropriate parameters such as duration or duty cycle. In addition, if different electrodes are available for delivery of stimulation, a therapy parameter of a therapy program may be further characterized by an electrode combination, which may define electrodes 24, 26 selected for delivery of electrical stimulation and their respective polarities. In some examples, stimulation may be delivered using a continuous waveform and the stimulation parameters may define this waveform.

In addition to being configured to deliver therapy to manage a disorder of patient 12, therapy system 10 may be configured to sense bioelectrical brain signals of patient 12. For example, IMD 16 may include a sensing module that is configured to sense bioelectrical brain signals within one or more regions of brain 28 via a subset of electrodes 24, 26, another set of electrodes, or both. Accordingly, in some examples, electrodes 24, 26 may be used to deliver electrical stimulation from the therapy module to target sites within brain 28 as well as sense brain signals within brain 28. However, IMD 16 can also use a separate set of sensing electrodes to sense the bioelectrical brain signals. In some examples, the sensing module of IMD 16 may sense bioelectrical brain signals via one or more of the electrodes 24, 26 that are also used to deliver electrical stimulation to brain 28. In other examples, one or more of electrodes 24, 26 may be used to sense bioelectrical brain signals while one or more different electrodes 24, 26 may be used to deliver electrical stimulation.

Examples of bioelectrical brain signals include, but are not limited to, electrical signals generated from local field potentials (LFPs) within one or more regions of brain 28, such as, but not limited to, an electroencephalogram (EEG) signal or an electrocorticogram (ECoG) signal. In some examples, the electrical signals within brain 28 may reflect changes in electrical current produced by the sum of electrical potential differences across brain tissue.

External medical device programmer 14 is configured to wirelessly communicate with IMD 16 as needed to provide or retrieve therapy information. Programmer 14 is an external computing device that the user, e.g., the clinician and/or patient 12, may use to communicate with IMD 16. For example, programmer 14 may be a clinician programmer that the clinician uses to communicate with IMD 16 and program one or more therapy programs for IMD 16. In addition, or instead, programmer 14 may be a patient programmer that allows patient 12 to select programs and/or view and modify therapy parameter values. The clinician programmer may include more programming features than the patient programmer. In other words, more complex or sensitive tasks may only be allowed by the clinician programmer to prevent an untrained patient from making undesired changes to IMD 16.

Programmer 14 may be a hand-held computing device with a display viewable by the user and an interface for providing input to programmer 14 (i.e., a user input mechanism). For example, programmer 14 may include a small display screen (e.g., a liquid crystal display (LCD) or a light emitting diode (LED) display) that presents information to the user. In addition, programmer 14 may include a touch screen display, keypad, buttons, a peripheral pointing device or another input mechanism that allows the user to navigate through the user interface of programmer 14 and provide input. If programmer 14 includes buttons and a keypad, then the buttons may be dedicated to performing a certain function, e.g., a power button, the buttons and the keypad may be soft keys that change in function depending upon the section of the user interface currently viewed by the user, or any combination thereof.

In other examples, programmer 14 may be a larger workstation or a separate application within another multi-function device, rather than a dedicated computing device. For example, the multi-function device may be a notebook computer, tablet computer, workstation, cellular phone, personal digital assistant or another computing device that may run an application that enables the computing device to operate as a secure medical device programmer 14. A wireless adapter coupled to the computing device may enable secure communication between the computing device and IMD 16.

When programmer 14 is configured for use by the clinician, programmer 14 may be used to transmit programming information to IMD 16. Programming information may include, for example, hardware information, such as the type of medical leads 20, the arrangement of electrodes 24, 26 on medical leads 20, the position of medical leads 20 within brain 28, one or more therapy programs defining therapy parameter values, and any other information that may be useful for programming into IMD 16. Programmer 14 may also be capable of completing functional tests (e.g., measuring the impedance of electrodes 24, 26 of medical leads 20).

With the aid of programmer 14 or another computing device, a clinician may select one or more therapy programs for therapy system 10 and, in some examples, store the therapy programs within IMD 16. Programmer 14 may assist the clinician in the creation/identification of therapy programs by providing physiologically relevant information specific to patient 12.

Programmer 14 may also be configured for use by patient 12. When configured as a patient programmer, programmer 14 may have limited functionality (compared to a clinician programmer) in order to prevent patient 12 from altering critical functions of IMD 16 or applications that may be detrimental to patient 12.

Whether programmer 14 is configured for clinician or patient use, programmer 14 is configured to communicate to IMD 16 and, optionally, another computing device, via wireless communication. Programmer 14, for example, may communicate via wireless communication with IMD 16 using radio frequency (RF) telemetry techniques known in the art. Programmer 14 may also communicate with another programmer or computing device via a wired or wireless connection using any of a variety of local wireless communication techniques, such as RF communication according to the 802.11 or BLUETOOTH® specification sets, infrared (IR) communication according to the IRDA specification set, or other standard or proprietary telemetry protocols. Programmer 14 may also communicate with other programming or computing devices via exchange of removable media, such as magnetic or optical disks, memory cards or memory sticks. Further, programmer 14 may communicate with IMD 16 and another programmer via remote telemetry techniques known in the art, communicating via a local area network (LAN), wide area network (WAN), public switched telephone network (PSTN), or cellular telephone network, for example.

Therapy system 10 may be implemented to provide chronic stimulation therapy to patient 12 over the course of several months or years. However, system 10 may also be employed on a trial basis to evaluate therapy before committing to full implantation. If implemented temporarily, some components of system 10 may not be implanted within patient 12. For example, patient 12 may be fitted with an external medical device, such as a trial stimulator, rather than IMD 16. The external medical device may be coupled to percutaneous medical leads or to implanted medical leads via a percutaneous extension. If the trial stimulator indicates DBS system 10 provides effective treatment to patient 12, the clinician may implant a chronic stimulator within patient 12 for relatively long-term treatment.

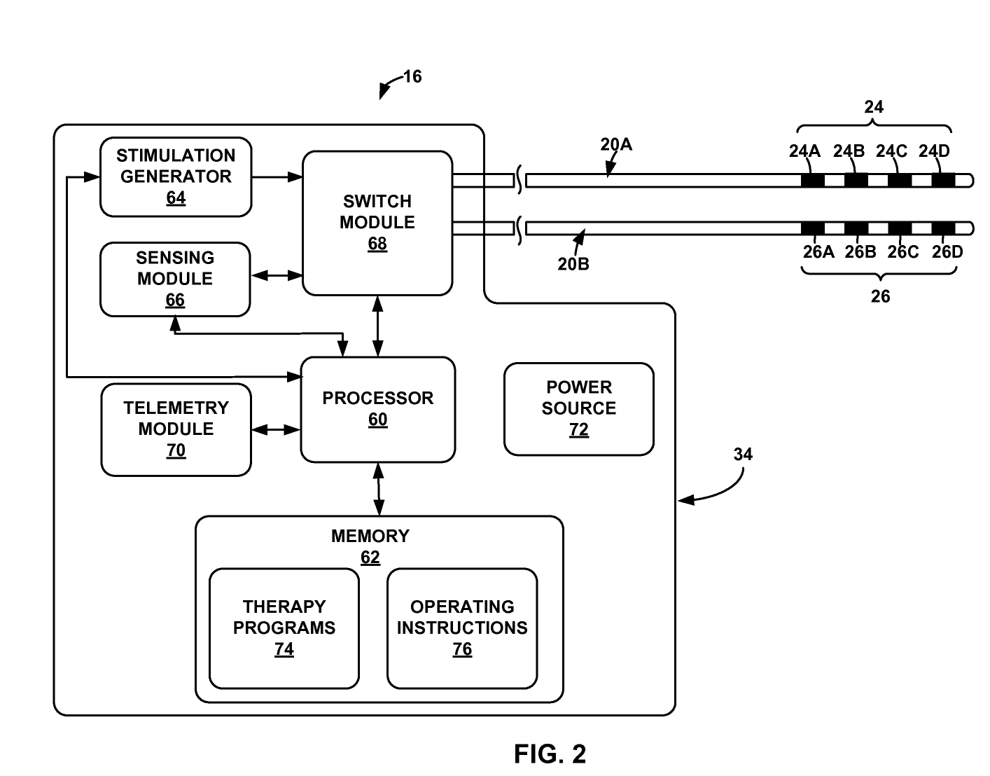

FIG. 2 is functional block diagram illustrating components of an example IMD 16. In the example shown in FIG. 2, IMD 16 includes processor 60, memory 62, stimulation generator 64, sensing module 66, switch module 68, telemetry module 70, and power source 72. Memory 62, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 62 may store computer-readable instructions that, when executed by processor 60, cause IMD 16 to perform various functions described herein.

In the example shown in FIG. 2, memory 62 stores therapy programs 74 and operating instructions 76, e.g., in separate memories within memory 62 or separate areas within memory 62. Each stored therapy program 74 defines a particular program of therapy in terms of respective values for electrical stimulation parameters, such as an electrode combination, current or voltage amplitude, and, if stimulation generator 64 generates and delivers stimulation pulses, the therapy programs may define values for a pulse width, and pulse rate of a stimulation signal. The stimulation signals delivered by IMD 16 may be of any form, such as stimulation pulses, continuous-wave signals (e.g., sine waves), or the like. Operating instructions 76 guide general operation of IMD 16 under control of processor 60, and may include instructions for monitoring brain signals within one or more brain regions via electrodes 24, 26 and delivering electrical stimulation therapy to patient 12.

Stimulation generator 64, under the control of processor 60, generates stimulation signals for delivery to patient 12 via selected combinations of electrodes 24, 26. In some examples, stimulation generator 64 generates and delivers stimulation signals to one or more target regions of brain 28 (FIG. 1), via a select combination of electrodes 24, 26, based on one or more stored therapy programs 74. The target tissue sites within brain 28 for stimulation signals or other types of therapy and stimulation parameter values may depend on the patient condition for which therapy system 10 is implemented to manage.

The processors described in this disclosure, including processor 60, may include one or more digital signal processors (DSPs), general-purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. Processor 60 is configured to control stimulation generator 64 according to therapy programs 74 stored by memory 62 to apply particular stimulation parameter values specified by one or more therapy programs.

In the example shown in FIG. 2, the set of electrodes 24 of medical lead 20A includes electrodes 24A, 24B, 24C, and 24D, and the set of electrodes 26 of medical lead 20B includes electrodes 26A, 26B, 26C, and 26D. Processor 60 may control switch module 68 to apply the stimulation signals generated by stimulation generator 64 to selected combinations of electrodes 24, 26. In particular, switch module 68 may couple stimulation signals to selected conductors within medical leads 20, which, in turn, deliver the stimulation signals across selected electrodes 24, 26. Switch module 68 may be a switch array, switch matrix, multiplexer, or any other type of switching module configured to selectively couple stimulation energy to selected electrodes 24, 26 and to selectively sense bioelectrical brain signals with selected electrodes 24, 26. Hence, stimulation generator 64 is coupled to electrodes 24, 26 via switch module 68 and conductors within medical leads 20. In some examples, however, IMD 16 does not include switch module 68. For example, IMD 16 may include multiple sources of stimulation energy (e.g., current sources).

Stimulation generator 64 may be a single channel or multi-channel stimulation generator. In particular, stimulation generator 64 may be capable of delivering a single stimulation pulse, multiple stimulation pulses or continuous signal at a given time via a single electrode combination or multiple stimulation pulses at a given time via multiple electrode combinations. In some examples, however, stimulation generator 64 and switch module 68 may be configured to deliver multiple channels on a time-interleaved basis. For example, switch module 68 may serve to time divide the output of stimulation generator 64 across different electrode combinations at different times to deliver multiple programs or channels of stimulation energy to patient 12. In other examples, stimulation generator 64 may provide independent stimulation sources for each of electrodes 24 and 26 such that any electrode may be used as a current source or sink in any combination with any other electrodes 24 and 26.

Sensing module 66, under the control of processor 60, is configured to sense bioelectrical brain signals of patient 12 via a selected subset of electrodes 24, 26 or with one or more electrodes 24, 26 and at least a portion of a conductive outer housing 34 of IMD 16, an electrode on outer housing 34 of IMD 16 or another reference. Processor 60 may control switch module 68 to electrically connect sensing module 66 to selected electrodes 24, 26. In this way, sensing module 66 may selectively sense bioelectrical brain signals with different combinations of electrodes 24, 26 (and/or a reference other than an electrode 24, 26). Although sensing module 66 is incorporated into a common housing 34 with stimulation generator 64 and processor 60 in FIG. 2, in other examples, sensing module 66 is in a separate outer housing from outer housing 34 of IMD 16 and communicates with processor 60 via wired or wireless communication techniques.

Telemetry module 70 is configured to support wireless communication between IMD 16 and an external programmer 14 or another computing device under the control of processor 60. Processor 60 of IMD 16 may receive, as updates to programs, values for various stimulation parameters from programmer 14 via telemetry module 70. The updates to the therapy programs may be stored within therapy programs 74 portion of memory 62. Telemetry module 70 in IMD 16, as well as telemetry modules in other devices and systems described herein, such as programmer 14, may accomplish communication by RF communication techniques. In addition, telemetry module 70 may communicate with external medical device programmer 14 via proximal inductive interaction of IMD 16 with programmer 14. Accordingly, telemetry module 70 may send information to external programmer 14 on a continuous basis, at periodic intervals, or upon request from IMD 16 or programmer 14.

Power source 72 delivers operating power to various components of IMD 16. Power source 72 may include a small rechargeable or non-rechargeable battery and a power generation circuit to produce the operating power. Recharging may be accomplished through proximal inductive interaction between an external charger and an inductive charging coil within IMD 16. In some examples, power requirements may be small enough to allow IMD 16 to utilize patient motion and implement a kinetic energy-scavenging device to trickle charge a rechargeable battery. In other examples, traditional batteries may be used for a limited period of time.

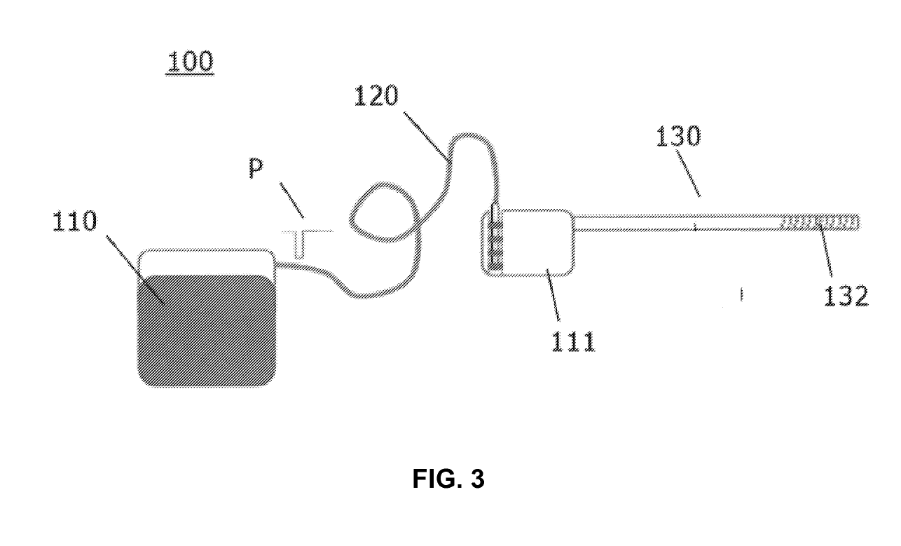

FIG. 3 is a functional block diagram illustrating components of an example neurostimulation system 100. System 100 of FIG. 3 includes implantable pulse generator (IPG) 110 (IPG may also be referred to as an IMD), which may include the pulse generation functionality of the system and sensing functionality, such as neural recording facilities. System 100 also includes active lead can (ALC) 111. IPG 110 connects to ALC 111 via interface cable 120, which may comprise multiple cables as discussed further below. In turn, ALC 111 connects to DBS lead 130 with a separate conductor for each of electrodes 132 via connector 520. ALC 111 includes electronic module 500 with an active switch matrix to direct stimulation from IPG 110 to any combination of electrodes 132. In some examples, ALC 111 may include a stimulation generator that is in addition to, or instead of, a stimulation generator that is provided within IPG 110 (e.g., stimulation generator 64 of FIG. 2), in which case switch matrix may direct stimulation provided by logic of the ALC 111 to any combination of electrodes. Likewise, the active switch matrix electronic module 500 can direct sensing signals from any combination of electrodes 132 to IPG 110. In some examples, ALC 111 may digitize sensing signals prior to sending them to IPG 110. IPG 110 may store the sensing signals or a subset of the sensing signals, analyze the sensing signals or a subset of the sensing signals, and/or forward the sensing signals or a subset of the sensing signals to an external device via a wireless transmission.

IPG 110 that may be surgically implanted in the chest region of a patient, such as below the clavicle or in the abdominal region of a patient. IPG 110 may be configured to supply the necessary electrical stimulation (e.g., voltage pulses). The neurostimulation system 100 may further include an extension wire 120 connected to IPG 110 and running subcutaneously to the skull, such as along the neck, where it terminates in a connector within ALC 111. Extension wire 120 may comprise a lead extension that has a connector at the proximal end that connects with a head block of IPG 110. The lead extension may further comprise a cable extending distally and terminating in a connector at a distal end. This distal end connector may be configured to mate with a connector at a proximal end cable of DBS lead 130. For instance, the DBS lead system may comprise a cable that is integrally formed with, and extends proximally from, ALC 111. This cable proximal to the ALC 111 may carry a connector at the proximal end that mates with the distal end connector of the lead extension. In this manner, it will be understood that extension wire 120 may comprise more than one cable, including a lead extension and an additional cable at the proximal end of ALC 111.

DBS lead 130 may be implanted in the brain tissue, e.g. through a burr-hole in the skull. In some examples, ALC 111 may be located adjacent the burr-hole and external to the skull and beneath the skin. In other examples, ALC 111 may be located into a surgeon-created recess adjacent the burr-hole in the skull and/or into the burr hole itself.

As illustrated, neurostimulation system 100 includes DBS lead 130 for brain applications with stimulation and/or recording electrodes 132, which may include forty electrodes 132 provided on an outer body surface at the distal end of DBS lead 130. However, the techniques described in this disclosure are not so limited. For instance, in some examples more or fewer than forty electrodes may be used.

IPG 110 may include more than one an implantable pulse generator for delivery of neurostimulation via electrodes 132, and/or one or more sensors configured to sense electrical fields within the brain of the patient, such as electrical fields representing a patient's brain activity and/or electrical fields created by delivery of DBS therapy. In examples in which IPG 110 includes both an implantable pulse generator and one or more sensors, in various examples, either the same set of electrodes or different sets of electrodes may be used for sensing as those used for DBS therapy. In some examples, ALC 111 may include one or more stimulation generators and/or sensors instead of/or in addition to, those provided by IPG 110.

By means of the extension wire 120 pulses P supplied by IPG 110, like stimulation pulses, can be transmitted to ALC 111. In other words, IPG 110 and ALC 111, illustrated in FIG. 3, combine to form an alternative to IMD 16, in which the functionality of IPG 110 and ALC 111 are contained within a single housing.

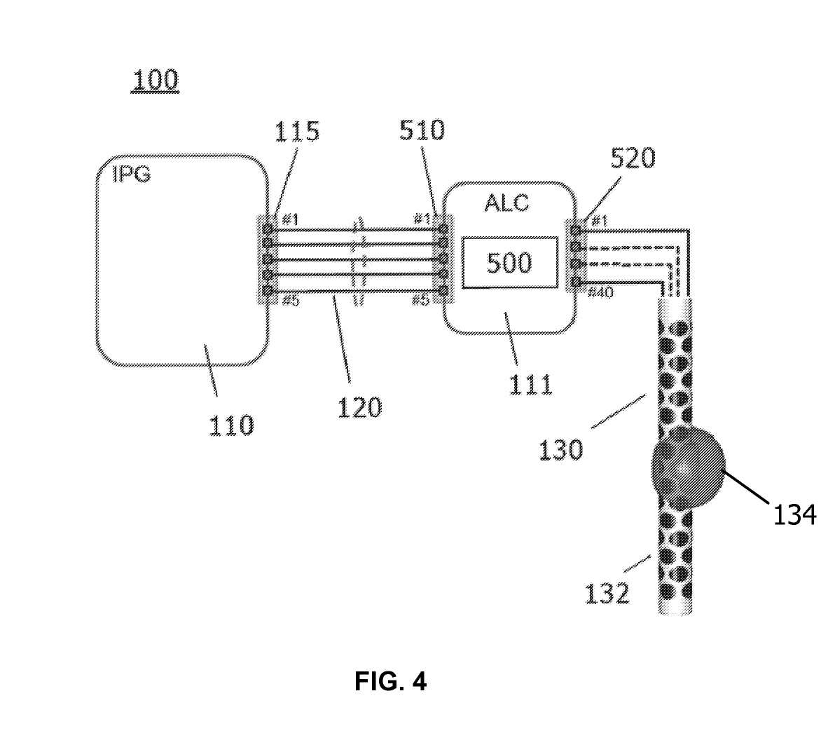

FIG. 4 is a functional block diagram illustrating electrical connections between IPG 100, ALC 111 and DBS lead 130 within neurostimulation system 100. As illustrated in FIG. 4, IPG 110 connects to ALC 111 via interface cable 120 and connectors 115, 510 respectively. In turn, ALC 111 connects to DBS lead 130 with a separate conductor for each of electrodes 132 via connector 520. ALC 111 includes electronic module 500 with an active switch matrix to direct stimulation from IPG 110 to any combination of electrodes 132. Likewise, the active switch matrix electronic module 500 can direct sensing signals from any combination of electrodes 132 to IPG 110. In the illustration of FIG. 4, example stimulation/sensing zone 134 is depicted. Stimulation/sensing zone 134 utilizes a subset of electrodes 132 for stimulation or sensing. The active switch matrix of electronic module 500 may be used to select any combination of electrodes for stimulation and sensing functionality. The switch matrix of electronic module 500 within ALC 111 can connect (directly or indirectly) any number of the available electrodes to any IPG line or ground. Such connections are not limited to being across pairs of two of electrodes 132. In one example, the connections between IPG 110 and electrodes 132 are indirect connections. For instance, intervening logic within the ALC 111 may provide signals to the electrodes 132 that are based, at least in part, on the signals received by the ALC 111 from IPG 110. In such an example, there is not necessarily any direct electrical connection between signal lines from the IPG 110 and electrodes 132. In this manner, various stimulation zones may be activated using different subsets of electrodes and/or by using field steering techniques such as varying the resistance of paths in multi-electrode combinations, anodal shielding and other field steering techniques.

In the current configuration, interface cable 120 and connectors 115, 510 provide five conductive paths between IPG 110 and ALC 111. This is in contrast to IMD 16 with medical leads 20A and 20B in that IMD 16 and medical leads 20A and 20B may provide a dedicated conductor between IMD 16 and each electrode. IPG 110 has a N-pin connector 115 (e.g., N=5) which is connected via the interface cable 120 with the 5-pin connector 510 of ALC 111. In the example of IPG 110 and ALC 111, the five conductors between IPG 110 and ALC 111 may include a power conductor, a ground conductor, a communication conductor, a conductor for a first pulse generator within IPG 110, and a conductor for a second pulse generator within IPG 110. The control line may provide instructions from IPG 110 for directing electrode pulses or sensing connectivity via the switch matrix to electronic module 500. In some examples, the power conductor may serve a dual purpose of providing clock or timing information between IPG 110 and ALC 111. For example, the voltage over the power conductor may be sent as a square wave or other periodic signal. In some examples, the timing information provided by the power conductor may be used to coordinate sensing and stimulation functions as isolating sensing circuitry from the stimulation generators may be required to protect the sensing circuitry from the stimulation pulse.

In some examples, ALC 111 includes a multi-pin connector with a 5-pin connector 510 for the interface cable 120 and a M-pin connector 520 (e.g., M=40) for DBS lead 130. These connectors may, or may not, be releasably (or selectively) connectable. For instance, in an example wherein connector 510 is not releasably-connectable, connector 510 may be integrally (semi-permanently) formed with a cable proximal to ALC 111. This cable is not disconnectable from connector 510. In such an example, interface cable 120 of FIG. 4 may comprise two cables: this first cable that is integrally coupled to connector 510 proximal to ALC 111 and a lead extension that mates with this ALC cable and further couples to IPG 110. In this case, a “disconnectable” connection is made between this proximal ALC cable and the lead extension rather than between a single cable 120 extending from IPG 110 and connector 510. In a similar manner, lead 130 may be integrally coupled to connector 520 in a manner that is non-releasable.

It is mechanically possible to design the two feed-through connectors 510, 520 with a high pin density to reduce the area of ALC 111 significantly. However, this area advantage may only materialize if the electrical components of ALC 111 are shrunk in similar proportions as the feed-through connectors 510, 520. Moreover, a very thin ALC 111, most desirable to reduce its impact on skin erosion, may need a high pin density, but also a reduction in the height of both feedthrough pins 511, 521 and interior electrical components. Thus, both the electronics volume and area of ALC 111 are miniaturized to realize a small ALC 111. Note that techniques to shrink ALC 111 can also be applied to the implantable pulse generator 110, or any other implant module, for example, to trade for an increase in battery life and/or increased functionality.

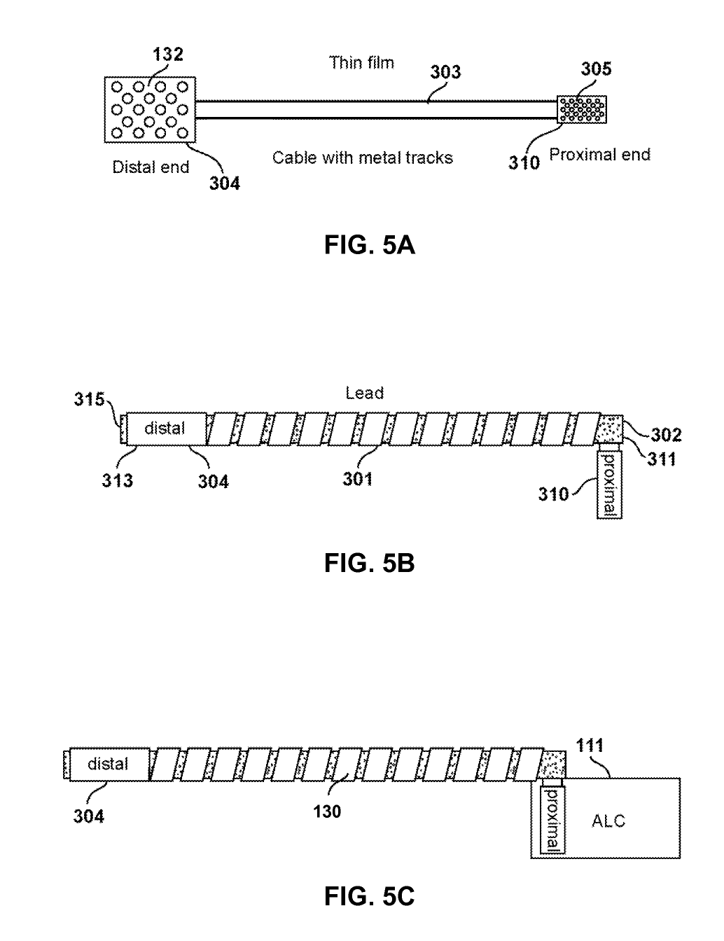

FIGS. 5A-5C illustrate examples of medical leads for stimulation and/or sensing. FIG. 5C further illustrates a typical architecture for an assembly including DBS lead 130 and ALC 111. ALC 111 includes an active switch matrix and electronics to address electrodes 132 on the distal end 304 of the thin film 301, which is arranged at the distal end 313 and next to the distal tip 315 of the DBS lead 130, as illustrated in FIG. 5B. The DBS lead 130 comprises a carrier 302 for a thin film 301, said carrier 302 providing the mechanical configuration of the DBS lead 130 and the thin film 301. Elongated carrier 302 may be a flexible carrier, such as a flexible tubing. In some examples, elongated carrier 302 may be formed from a silicone tubing.

Elongated carrier 302 may have any suitable configuration. In some examples, elongated carrier 302 may be an elongated member having a circular cross-section, although other cross-sections are contemplated, such as, e.g., square or hexagonal. Elongated carrier 302 may be a solid member or have a hollow core. In some examples, it is preferred that elongated carrier 302 be relatively stiff during implantation but able to flex or bend to some degree after implantation. The hollow core may allow for the insertion of a stiffening member such as a stylet into the hollow core, e.g., during implantation of lead 300. Elongated carrier 302 may be configured to not substantially shrink, stretch, or compress during and/or after implantation.

In some examples, elongated carrier 302 should be flexible and have a good rotational torque transfer, e.g., in instances of permanent (chronic) implant of lead 300. Some acute applications may have a different set of preferences. For instance, in acute implantation, no burr-hole devise may be used and flexibility and limited compressibility are of less concern.

Elongated carrier 302 may be formed of any suitable material including silicone, titanium, and/or polyether ether ketone (PEEK) based materials. For the mechanical requirements as mentioned above, other polymers can be more useful, e.g., bionate. In addition, metal tubes (e.g., laser machined to bendable chains) may be used. In acute applications, a solid metal may be used for elongated carrier 302. In acute application, there may not be a need for elongated carrier 302 to be hollow or flexible. In chronic applications, elongated carrier 302 is implanted with a stiffener inside. After implantation, the stiffener may be removed.

Distal portion of lead 300 may have a diameter between about 0.5 millimeters (mm) and about 3 mm diameter, e.g., about 1.3 mm. The diameter of lead 300 may be defined by the diameter of carrier core 302 in combination with the thickness of thin film 301 and any coating applied over carrier core 302 and/or thin film 301. The proximal portion of lead 300 (the portion adjacent to ALC 111) may have a diameter between about 0.5 mm and about 4 mm diameter. The length of lead 300 may be about 10 centimeters (cm) to about 20 cm, e.g., about 15 cm, and may vary based on the particular application, e.g., acute versus chronic implantation. Other dimensions than those examples described herein are contemplated.

The thin film 301 may include at least one electrically conductive layer, such as one made of a biocompatible material. The thin film 301 is assembled to the carrier 302 and further processed to constitute the DBS lead 130. The thin film 301 for a medical lead may be formed by a thin film product having a distal end 304, a cable 303 with conductive (e.g., metal) tracks and a proximal end 310, as illustrated in FIG. 5A. The proximal end 310 of the thin film 301 arranged at the proximal end 311 of the DBS lead 130 is electrically connected to ALC 111. ALC 111 comprises the switch matrix of the DBS steering electronics. The distal end 304 comprises the electrodes 132 for the brain stimulation. The proximal end 310 comprises the interconnect contacts 305 for each metal line in the cable 303. The cable 303 comprises metal lines (not shown) to connect each of distal electrodes 132 to a designated proximal contact 305.

DBS leads may be directed towards a relatively small target in brain, and precisely locating a DBS lead may be required to facilitate sensing and/or DBS therapies. Various tooling may be used, such as software planning, stereo tactic frames, for proper positioning, but all tools and processes expect the medical lead itself to be straight. A DBS leads itself may be flexible to compensate for brain tissue movement.

During a DBS implantation procedure, a DBS lead may be temporarily stiffened by inserting a stylet in the core of the DBS lead. After positioning of the DBS lead, the stylet is removed. Even a DBS lead stiffened with a stylet is not completely stiff; with some sideways exercised force, the DBS lead bends during implantation. This can cause the DBS lead to deviate from its planned trajectory, while moving towards the intended target. Similarly, bending may occur when a guide catheter or other lead delivery system is used of implant a lead in a tissue of a patient. As this deviation is hard to detect, a mechanism to determine the DBS lead bending along the DBS lead may improve the precision of the DBS lead implantation procedure. This mechanism to determine bending of the lead is discussed in detail below.

In one particular example, a DBS lead such as shown in FIG. 1 may include, e.g., four 1.5 millimeters-wide cylindrical electrodes at the distal end spaced by between about 0.5 millimeters and 1.5 millimeters. In this example, the diameter of the medical lead is may be about 1.27 millimeters and the metal used for the electrodes and the interconnect wires may be an alloy of platinum and iridium. The coiled interconnect wires may be insulated individually by fluoropolymer coating and protected in an 80 micron urethane tubing. With such an electrode design, the current distribution may emanate uniformly around the circumference of the electrode, which medical leads to stimulation of all areas surrounding the electrode.

Such a design may limit fine spatial control over stimulation field distributions. The lack of fine spatial control over field distributions implies that stimulation easily spreads into adjacent structures inducing adverse side effects in about thirty percent of the patients. To overcome this problem, medical leads with high density electrode arrangements, such as those examples illustrated in FIGS. 5A-11 herein and/or leads having segmented ring electrodes such as discussed above, facilitate electrical field position adjustments in smaller increments, hence providing the ability to steer the stimulation field to the appropriate target.

The clinical benefit of DBS may be largely dependent on the spatial distribution of the stimulation field in relation to brain anatomy. To improve efficacy and efficiency of DBS while avoiding unwanted side effects, precise control over the stimulation field is important.

DBS leads may implement monopolar, bipolar, or even tripolar stimulation. Neurostimulator devices with steering brain stimulation capabilities may have a large number of electrode contacts (n>10) that may be connected to electrical circuits such as current sources, voltage sources, and/or (system) ground. Stimulation may be considered monopolar when the distance between the anode and cathode is several times larger than the distance of the cathode to the stimulation target. During monopolar stimulation in homogeneous tissue, the electric field may be distributed roughly spherically similar to the field from a point source. When the anode is located close to the cathode, creating a bipolar electrode combination, the distribution of the field becomes more directed in the anode-cathode direction. As a result, the field gets stronger and neurons may be more likely to be activated in this area due to a higher field gradient.

Polarization (de- and/or hyperpolarization) of neural tissue may play a prominent role for both suppression of clinical symptoms, as well as induction of stimulation-induced side effects. In order to activate a neuron, it has to be depolarized. Neurons may be depolarized more easily close to the cathode than by the anode (about 3-7 times more depending on type of neuron, etc.).

With a very small target in the lower brain, e.g. the subthalamic nucleus (STN), targeting for example a DBS lead into its exact location is not trivial. Various mechanisms are used like software planning tools, stereo tactic frames and the like for proper positioning, but all tools and processes may expect the medical lead itself to be straight. The medical lead is, however, to a certain extent flexible, e.g., to compensate brain tissue movement. With some sideways exercised force, the medical lead may bend. This may cause the medical lead to deviate from its planned trajectory, while moving towards the intended target (e.g. the STN). As this deviation is hard to detect, as disclosed herein, a bending sensor may be used to improve the implantation. In this example, the bending sensor measures the bending of the medical lead, e.g., across the whole length of the medical lead or across a portion of the medical lead and potentially may determine the bending direction.

When bending is known across a predetermined portion of the length of the medical lead, e.g., the portion extending from a burr hole in the patient's skull to the distal lead tip or substantially the entire length of the lead, one may also derive the exact position of the medical lead in the brain, including the position of the distal end of the medical lead with the electrodes. Thus, the accuracy of the implantation of neurostimulation medical leads may be significantly improved as compared to implantation procedures in which bending of the medical lead is not evaluated.

In some examples, the bending sensor may be integrated with or carried by a lead delivery device used to implant the medical lead. In some examples, the lead delivery device may include a stylet, a guide catheter, or any other known type of lead delivery system. For example, prior to the implantation of a lead, a stylet may be inserted within a hollow core of the lead to increase the rigidity of the lead during implant. In the case of a stylet, the bending system may be integrated in or on the stylet being temporarily insertable into the medical lead during implantation of the lead. Such a stylet may be used during implantation to stiffen the medical lead. The stylet is only used during implantation and thus the bending sensor may be removed together with the stylet and reused.

As another example of a lead delivery device, a guide catheter may be used in which the lead inserted within a hollow core of the catheter to be implanted, e.g., after the guide catheter has been implanted in the tissue of a patient and/or prior to the implantation such that the combination of the catheter and lead is implanted in a patient. Once the lead is in place, the guide catheter may be removed from the patient. In the case of a guide catheter, the bending system may be integrated in or on the guide catheter being temporarily implanted in a patient during implantation of the lead. Such a guide catheter may be used during implantation to stiffen the medical lead. The guide catheter is only used during implantation and thus the bending sensor may be removed together with the guide catheter and reused.

Alternatively or additionally, the bending sensor may be integrated into the medical lead itself. By this, bending of the medical lead after implantation may also be measured, as the medical lead might be subject to forces that may be, e.g., caused by movements of the brain or forces outside of the brain.

In some examples, the bending sensor may comprise or be at least one optical sensor. Optical sensors allow an easy implementation into either a lead delivery device (e.g., stylet or guide catheter) and/or the medical lead, need small space to be implemented and allow a bending measurement with high accuracy.

In some examples, as an optical sensor, the bending sensor may comprise an optical fiber. For example, light signals or laser signals may be sent into the optical fiber and the runtime or reflection patterns may be used to determine the bending, as, e.g., runtime or reflection changes depending on the bending of the optical fiber. For example, the optical sensor may include an optical fiber with Bragg grating(s). In some examples, the output of the optical sensor may provide both the direction and amplitude of the bending. The optical fiber may extend on or within a guide catheter, stylet (or other lead delivery device), or the lead itself to sense bending during implant of the lead. In some example, the body of a guide catheter, stylet (or other lead delivery device), or the lead itself may be formed of an optical fiber.

In some examples, the bending sensor may comprise or be at least one piezoelectric sensor. The use of piezoelectric sensors is a further additional or alternative option to measure the bending. Piezoelectric sensors may be also well established and need not require much space for implementation. The piezoelectric resistive effect may, e.g., cause resistance changes, which may be measured and related to the bending and the direction of the bending of the medical lead. With two or more piezoelectric sensors, such as two sensors 90 degrees apart, the output of the piezoelectric sensors may provide both the direction and amplitude of the bending.

In some examples, the bending sensor may comprise or be at least one resistance sensor, which changes its path length due to the bending. This is a further additional or alternative solution, for how to measure the bending of the medical lead within small space and high accuracy. By adding e.g. a resistive path or more resistive paths on the medical lead or the stylet (for example a plurality of thin film tracks), the path length may change, if the medical lead, stylet, guide catheter and/or other lead delivery device bends. By measuring the resistance changes, the bending and the direction of this bending may be determined. With two or more resistance sensors, such as two sensors 90 degrees apart, the output of the resistance sensors may provide both the direction and amplitude of the bending.

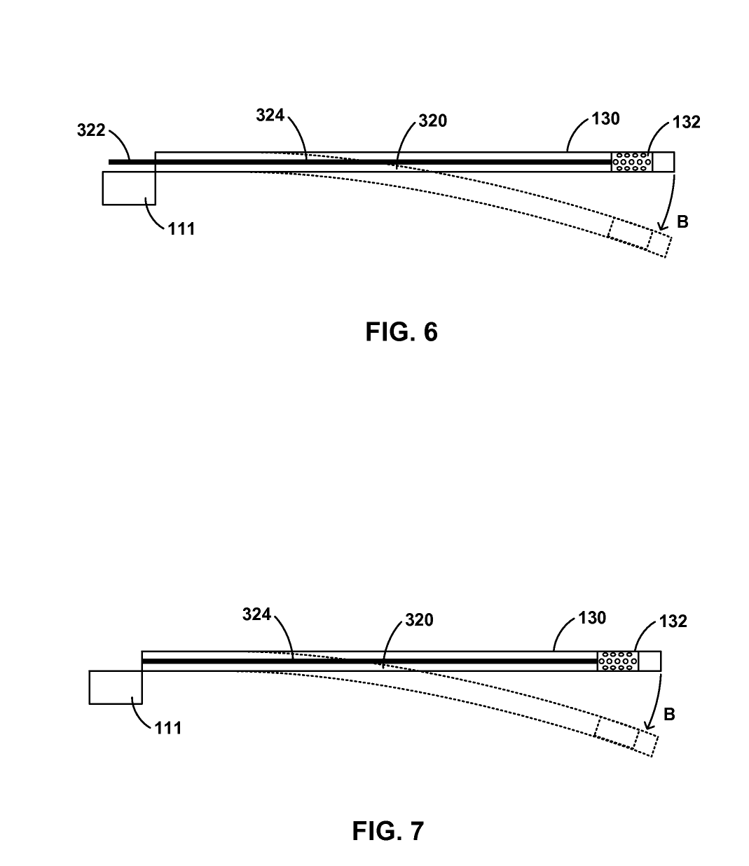

Such examples are described in further detail with respect to FIGS. 6-11.

FIG. 6 illustrates a medical device system for neurostimulation and neurorecording, including DBS lead 130 and the bending sensor 320, which includes stylet 322. For ease of description, examples of the disclosure employing a lead delivery device are described primarily with regard to the lead delivery device taking the form of a stylet. However, examples of lead delivery device are not limited to stylet but may include, e.g., any lead delivery device known for the implantation of medical lead into a tissue of a patient, such as, e.g., a guide catheter and the like.

In example of FIG. 6, the bending sensor 320 is integrated into the stylet 322. In the example of FIG. 6 the bending sensor is an optical sensor comprising an optical fiber 324, e.g., disposed inside an inner lumen of the stylet. The sensor may also be an integral part of the stylet itself, rather than within a lumen of the stylet. The configuration of the optical sensor and stylet is such that bending of the stylet is transferred to the optical sensor in a predictable manner such that bending measurements within the optical sensor are representative to bending of the stylet.

In the example shown, bending of lead 130 which is distal to ALC 111 is determined. This may be the portion of the lead system that is implanted within brain tissue. It may not be necessary to determine a bend in at least one other portion of the lead system, such as a portion of the system proximal to ALC 111 (e.g., a cable extending proximal to ALC 111). This is because such a portion proximal to ALC 111 may be adapted to run under the scalp next to the skull and is not implanted in the brain. Therefore, determining the bend of this other portion may not be needed.

DBS lead 130 may have an outer diameter of less than approximately 3 millimeters, and especially less than approximately 2.5 millimeters. In some examples, DBS lead 130 may be thicker adjacent ALC 111 than at its distal end due to reinforcement structures within those areas. Stylet 322 may have an outer diameter of less than approximately 1.5 millimeters, such as less than approximately 1 millimeter, such as less than approximately 0.5 millimeters, such as between about 0.3 millimeters to 0.4 millimeters.

The following functions may be provided by the system 100 according to the present disclosure. With a tiny target in the lower brain, e.g. the subthalamic nucleus (STN), targeting for example a DBS lead into its exact location is not trivial. Various tooling is used like software planning tools, stereo tactic frames and the like for proper positioning, but all tools and processes expect the DBS lead 130 itself to be straight. The DBS lead 130 is, however, to a certain extent flexible, e.g. to compensate brain tissue movement.

During the medical lead implantation procedure, DBS lead 130 may be temporarily stiffened by inserting the stylet 322 in a core of the DBS lead 130, e.g., inside an inner lumen of the lead. After positioning of the DBS lead 130, the stylet 322 is removed.

Even a stiffened DBS lead 130 may not be completely stiff, with some sideways exercised force, the DBS lead 130 may bend. This may cause the DBS lead 130 to deviate from its planned trajectory, while moving towards the intended target (e.g. the STN). As this deviation is hard to detect, a bending detection may be needed to improve the implantation.

This bending detection is provided by the bending sensor 320. In this example, the bending sensor 320 measures the bending B of the DBS lead 130 across the whole length of the DBS lead 130 and the bending direction. When bending is known across the complete length of the DBS lead 130, one may also derive the exact position of the DBS lead 130 in the brain, including the position of the distal end of the DBS lead 130 with the electrodes 132, because the DBS lead 130 is referenced with respect to the stereotactic frame.

By using the optic fiber 324 that runs along the stylet 322, the bending of the DBS lead 130 may be determined, e.g., regarding position and amount of bending along the length of the DBS lead 130, e.g., based on the analysis of light reflection spectrum patterns generated by, e.g., a laser signal generating source. This optic fiber approach provides all information including the bending along the length of the medical lead and the exact position of the array of electrodes 132 at the distal end of the DBS lead 130.

FIG. 7 is a conceptual illustration of another example system for neurostimulation and/or neurorecording with a DBS lead 130 and bending sensor 320. The example of FIG. 7 may include the structural and functional elements of the example of FIG. 6, however with the following difference: the bending sensor 320 is integrated into the DBS lead 130 rather than in a stylet. As with the system of FIG. 6, bending sensor 320 includes an optical sensor comprising at least one optical fiber 324. In some examples, both stylet 322 and lead 130 may include bending sensor 320 which each may be used to detect bending of lead 130 during implant.

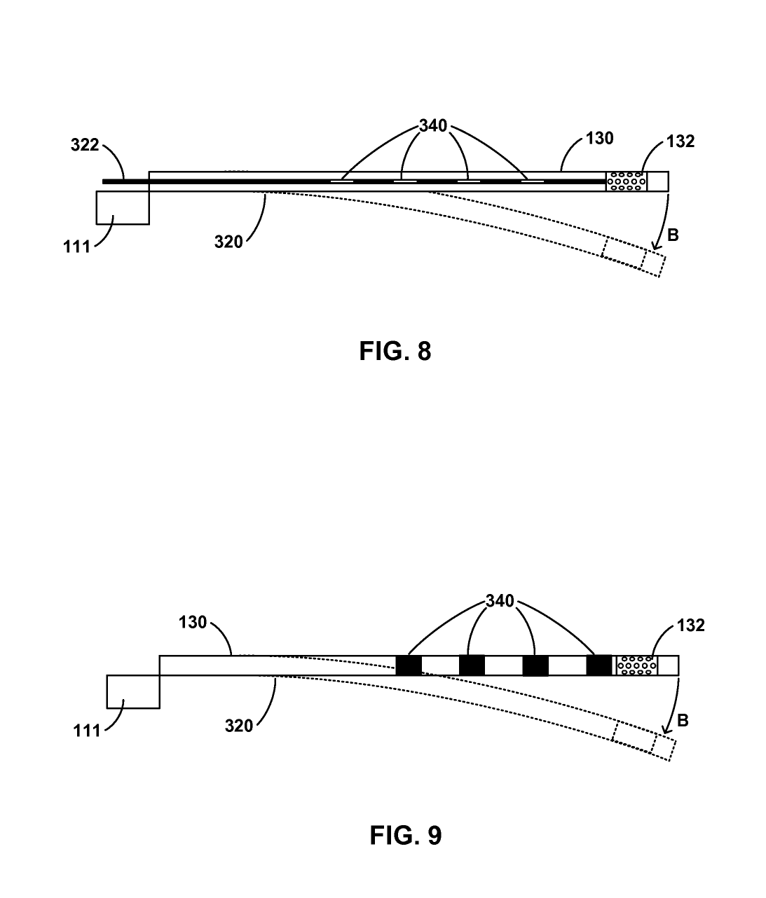

FIG. 8 is a conceptual illustration of another example system for neurostimulation and/or neurorecording with a DBS lead 130 and bending sensor 340. The example of FIG. 8 may include the structural and functional elements of the example of FIG. 6, except that the bending sensor is a piezoelectric sensor 340 that is integrated into the stylet 322. There may be a plurality of piezoelectric sensors 340 as shown in FIG. 8. The piezoelectric resistive effect may, e.g., cause resistance and/or voltage changes, which may be measured and related to the bending and the direction of the bending of the DBS lead 130.

FIG. 9 is a conceptual illustration of another example system for neurostimulation and/or neurorecording with a DBS lead 130 and bending sensor 340. The example of FIG. 9 includes each and every structural and functional feature of the example of FIG. 6, except that the bending sensor is a piezoelectric sensor 340 that is integrated into the DBS lead 130. In some examples, both stylet 322 and lead 130 may include bending sensor 320 which each may be used to detect bending of lead 130 during implant.

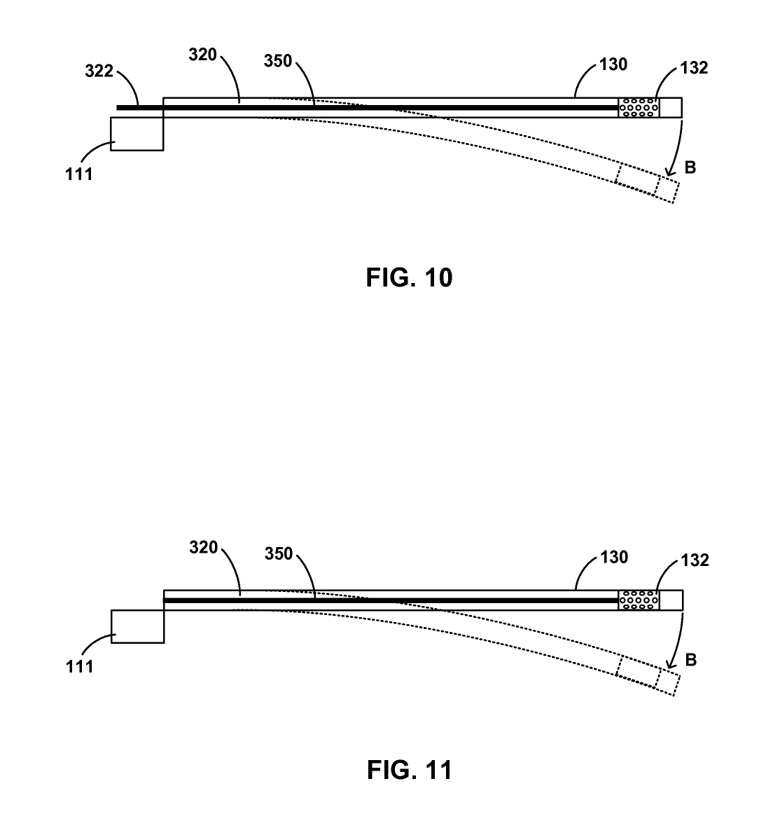

FIG. 10 is a conceptual illustration of another example system for neurostimulation and/or neurorecording with a DBS lead 130 and bending sensor 350. The example of FIG. 10 may include the structural and functional elements of the example of FIG. 6, except that the bending sensor is a resistance sensor 350. Resistance sensor 350 changes its path length and conductive area due to the bending of the medical lead. For example, with a resistance sensor with fractured microscopic structures, bending may leads to more connections between the individual microscopic resistors resulting in lower overall resistance.

In the example of FIG. 10, resistance sensor is integrated within stylet 322. By adding, e.g., a resistive path or more resistive paths on stylet 322, such as, for example, a plurality of thin film tracks, the path length may change, due to bending of the lead and stylet assembly. By measuring the resistance changes of two or more resistance sensing elements, such as two sensors 90 degrees apart, the bending and the direction of this bending may be determined to facilitate precise bending measurements of DBS lead 130.

FIG. 11 is a conceptual illustration of another example system for neurostimulation and/or neurorecording with a DBS lead 130 and bending sensor 350. The example of FIG. 11 may include the structural and functional elements of the example of FIG. 6, except that the bending sensor is a resistance sensor 350 integrated into DBS lead 130. Resistance sensor 350 changes its path length due to the bending of DBS lead 130. In the example of FIG. 10, resistance sensor is integrated within DBS lead 130. By adding e.g. a resistive path or more resistive paths on DBS lead 130, such as, for example a plurality of thin film tracks, the path length may change, due to bending of DBS lead 130. By measuring the resistance changes, the bending and the direction of this bending may be determined to facilitate precise bending measurements of DBS lead 130. In some examples, both stylet 322 and lead 130 may include bending sensor 320 which each may be used to detect bending of lead 130 during implant.

As described herein, in some examples, the sensors may be incorporated into another type of delivery system. For instance, a guide catheter may be provided to guide DBS lead 130 into position. One or more bending sensors may be located on or within the walls of guide catheter and used to determine the bend of the lead, e.g., in the case of a resistance sensor, piezoelectric sensor, or optical fiber. In some examples, the guide catheter body may be formed of an optical fiber which functions at the bending sensor, e.g., in the case of a hollow optical fiber tube.

The medical leads and bending sensors described with respect to FIGS. 6-11 may be modified within the spirit of this disclosure. For example, bending sensors of any suitable configuration located in a manner that facilitates evaluation of the bending of the medical lead during implantation may be used.

As one example, a capacitive sensor could be used as a bending sensor in conjunction with a lead or stylet. In some particular examples, a capacitive sensor may be integrated on a flexible film along the guide catheter/stylet/lead that has a capacitor structure, such as a short strip with interlaced fingers, whose capacitance changes with the bending direction. For example, with a capacitive sensor, compression may lead to higher capacitance and expansion may lead to lower capacitance. Two or more capacitive sensors, such as two sensors 90 degrees apart, provides information of the bending in the x, y directions at the position the strips are applied to allow for determination of the magnitude and amplitude of the bending.

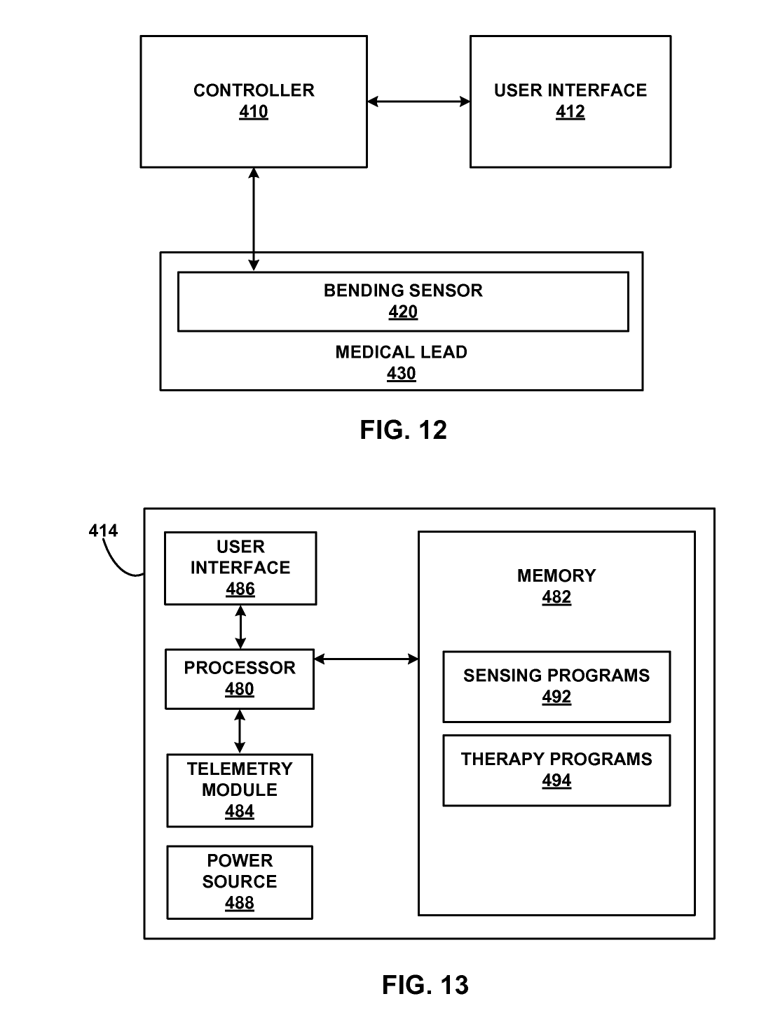

FIG. 12 is a functional block diagram illustrating components of an example system including medical lead 430, bending sensor 420 and controller 410. In some examples, medical lead 430 may be a DBS lead, such a DBS lead 130. In the same or different examples, bending sensor may include optical sensor, a piezoelectric sensor, or a resistance sensor, as previously described. In addition, the bending sensor may be located within medical lead 430 itself, or within a stylet used to facilitate implantation of the medical lead, or within a guide catheter or within any other type of lead delivery system known in the art, as previously described.

Controller 410 is configured to monitor a signal from bending sensor 420 and evaluate bending of medical lead 430 during the insertion based on the monitored signal. Controller 410 is also configured to generate information to guide steering of the medical lead toward a target site based on the evaluation of the bending of the medical lead. In some examples, controller 410 may present the information to a clinician via user interface 412 to allow the clinician to monitor the bending of medical lead 430 and potentially adjust the implantation. For example, user interface 412 may include a display or speaker that relays the generated information to the clinician. In the same or different examples, controller 410 may send the generated information to a system used to automatically guide the implantation of medical lead 430. For example, the generated information could be used as an input to guide the motion of the lead insertion tool of a stereotactic frame mounted to the patient to facilitate precise positioning of medical lead 430 within a tissue of the patient, such as the brain of the patient.

In one specific example, controller 410 comprises a dedicated computing device that connects to bending sensor 420 only during an implantation procedure. For example, the dedicated computing device may connect to an electomechanical lead insertion apparatus in which the control of the lead is based on feedback from the sensed bending of the lead to allow closed-loop control of the positioning of the lead during the implantation procedure. In the same or other examples, sensed lead bending information may be output to a user, such as the surgeon.

In other examples, the functionality of controller 410 or a portion thereof may be embodied within a component of a neurostimulation or neurosensing system, such as IMD 16 or IPG 111. In the same or different examples, a portion thereof may be embodied within a programmer for a neurostimulation or neurosensing system, such as medical device programmer 414.

Controller 410 may include one or more processor to allow controller 410 to function as described herein. The one or more processors may include one or more digital signal processors (DSPs), general-purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. In some examples, controller 410 may include or take the form of an external programmer device, such as, e.g., programmer 414.

FIG. 13 is a functional block diagram illustrating components of an example medical device programmer 414. Programmer 414 includes processor 480, memory 482, telemetry module 484, user interface 486, and power source 488. Processor 480 controls user interface 486 and telemetry module 484, and stores and retrieves information and instructions to and from memory 482. Programmer 414 may be configured for use as a clinician programmer or a patient programmer. Processor 480 may comprise any combination of one or more processors including one or more microprocessors, DSPs, ASICs, FPGAs, or other equivalent integrated or discrete logic circuitry. Accordingly, processor 480 may include any suitable structure, whether in hardware, software, firmware, or any combination thereof, to perform the functions ascribed herein to processor 480 and programmer 414.

A user, such as a clinician or patient 12, may interact with programmer 414 through user interface 486. User interface 486 includes a display (not shown), such as a LCD or LED display or other type of screen, with which processor 480 may present information related to the therapy (e.g., therapy programs,). In addition, user interface 486 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processor 480 of programmer 414 and provide input. In other examples, user interface 486 also includes audio circuitry for providing audible notifications, instructions or other sounds to patient 12, receiving voice commands from patient 12, or both.

Memory 482 may include instructions for operating user interface 486 and telemetry module 484, and for managing power source 488. Processor 480 may store the therapy programs and in memory 482 as stored therapy programs 494 and store the sensing parameters and the recorded results of the sensing as stored sensing programs 492. A clinician may review the stored therapy programs 494 and stored sensing programs 492 (e.g., during programming of IMD 16) to select one or more therapy programs with which IMD 16 may deliver efficacious electrical stimulation to patient 12. For example, the clinician may interact with user interface 486 to retrieve the stored therapy programs 494 and stored sensing programs 492.

In some examples, processor 480 is configured to generate and present, via a display of user interface 486, a graphical user interface (GUI) that presents a list of therapy programs. A user (e.g., a clinician) may interact with the GUI to manipulate the list of therapy programs. In some examples, a user may also interact with the graphical user interface to select a particular therapy program, and, in response to receiving the user input, programmer 414 may provide additional details about the therapy program. For example, the additional details presented by programmer 414 may include details about the individual parameter settings of the therapy program, such as the electrical stimulation parameter values, electrode combination, or both.

In some examples, patient 12, a clinician or another user may interact with user interface 486 of programmer 414 in other ways to manually select programs from the stored therapy programs 494 and stored sensing programs 492 for programming IMD 16, generate new therapy and sensing programs, modify stored therapy programs 494 and stored sensing programs 492, transmit the selected, modified, or new programs to IMD 16, or any combination thereof.

Memory 482 may include any volatile or nonvolatile memory, such as RAM, ROM, EEPROM or flash memory. Memory 482 may also include a removable memory portion that may be used to provide memory updates or increases in memory capacities. A removable memory may also allow sensitive patient data to be removed before programmer 414 is used by a different patient.

Wireless telemetry in programmer 414 may be accomplished by RF communication or proximal inductive interaction of external programmer 414 with IMD 16. This wireless communication is possible through the use of telemetry module 484. Accordingly, telemetry module 484 may be similar to the telemetry module contained within IMD 16. In other examples, programmer 414 may be capable of infrared communication or direct communication through a wired connection. In this manner, other external devices may be capable of communicating with programmer 414 without needing to establish a secure wireless connection.

Power source 488 is configured to deliver operating power to the components of programmer 414. Power source 488 may include a battery and a power generation circuit to produce the operating power. In some examples, the battery may be rechargeable to allow extended operation. In other examples, traditional batteries (e.g., nickel cadmium or lithium ion batteries) may be used. In addition, programmer 414 may be directly coupled to an alternating current outlet to operate.

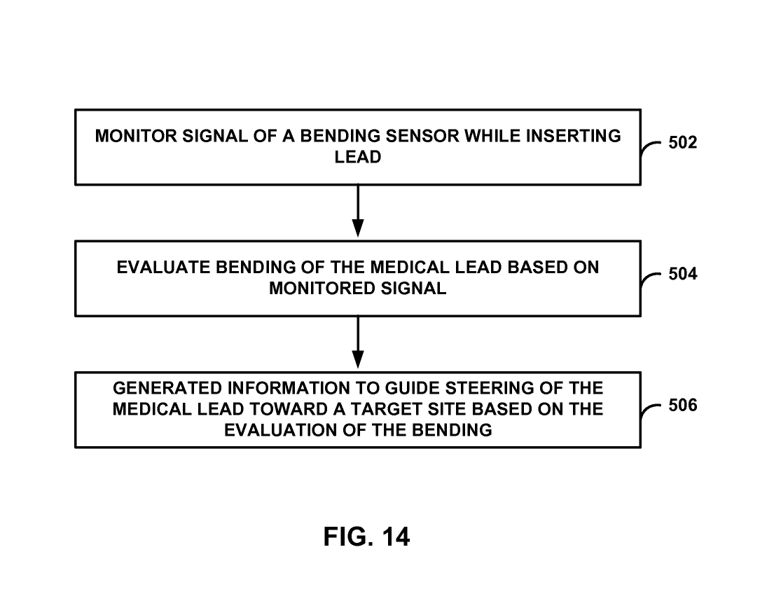

FIG. 14 is a flowchart illustrating an example technique for guiding implantation of a medical lead including monitoring a signal from a bending sensor associated with the medical lead while the medical lead is being inserted into tissue of a patient. For clarity, the techniques of FIG. 14 are described with respect to the medical device system illustrated in FIG. 12 although such techniques may be employed by any suitable system.

As shown, while medical lead 430 is being inserted into tissue of a patient, controller 410 monitoring a signal from bending sensor 420, which is associated with medical lead 430 (502). While medical lead 430 is being inserted into tissue of a patient, controller 410 evaluates bending of the medical lead during the insertion based on the monitored signal (504). In some examples, controller 410 may also monitor the signal from bending sensor 420 prior to implant of lead 430, e.g., to provide a baseline output signal that may be used for comparison to the sensor output during the implant process to detect bending. Controller 410 may determine that lead 430 is bending some amount (e.g., from some known non-bent state of lead 430) and/or may also determine the actual amount of bending of lead 430 based on the signal. Controller 410 may make such a determination, e.g., by comparing the output of the signal during the implantation to a baseline sensor output reflective of lead 430 in a non-bent state and/or other sensor outputs that have been determined to correspond the bending of lead 410 in some known amount. Other suitable techniques for evaluating the bending of lead 430 during implant based on the output of bending sensor 420 may be employed.

While medical lead 430 is being inserted into tissue of a patient, controller 410 generates information to guide steering of the medical lead toward a target site based on the evaluation of the bending of the medical lead (506). For example, controller 410 may present the information to a clinician via user interface 412 to allow the clinician to monitor the bending of medical lead 430 and potentially adjust the implantation. For example, user interface 412 may include a display or speaker that relays the generated information to the clinician. In some cases, the user interface may provide a display including medical lead 430 that is overlaid with patient anatomical data to allow the clinician to determine how the lead is bending with respect to the anatomy. This may help the clinician adjust the implantation based on this bending.

A clinician may monitor an output representative of the bending sensor signal and manually adjust the insertion force and direction of the lead to reduce the bending to the extent practical. In this manner, the medical lead may be manually directed in a generally linear direction by the clinician such that placement of the medical lead is according to the original insertion location and originally planned trajectory. In the same or different examples, the information generated by controller 410 may be used as an input to guide the motion of the lead insertion tool of a stereotactic frame mounted to the patient to facilitate precise positioning of medical lead 430 within a tissue of the patient, such as the brain of the patient.

In some examples, inserting the medical lead within a tissue of a patient to locate the medical lead adjacent a target site within the patient includes inserting the medical lead in combination with a stylet, guide catheter, or other lead delivery device inserted into the medical lead within the tissue of the patient. In such examples, the bending sensor may be integrated in the stylet, guide catheter, or other lead delivery device, for example, as illustrated in the examples of FIGS. 6, 8 and 10 for a stylet. Additionally or alternatively, in some examples, the bending sensor may be integrated into the medical lead, for example, as illustrated in the examples of FIGS. 7, 9 and 11.

In some examples, the method may further include, after locating the medical lead adjacent the target site within the patient, delivering electrical stimulation therapy via the medical lead. In the same or different examples, the method may further include, after locating the medical lead adjacent the target site within the patient, sensing of electrical fields within the brain of the patient via the medical lead.

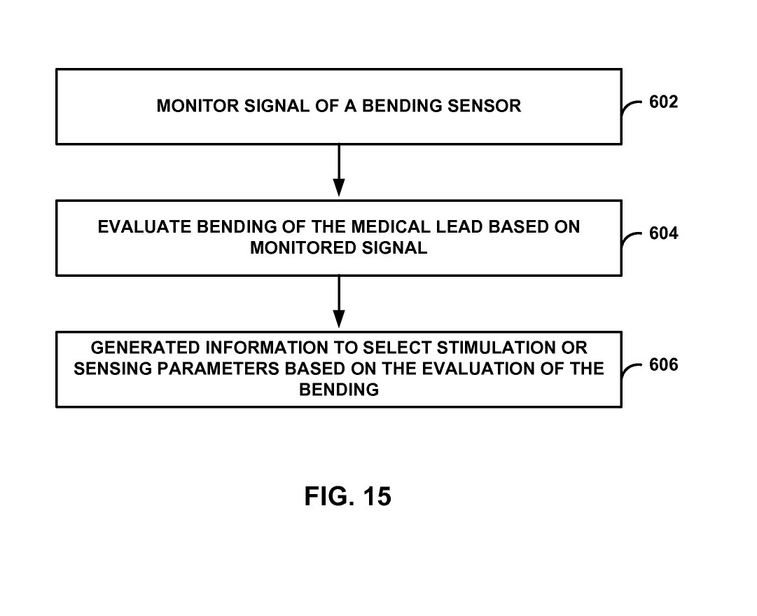

FIG. 15 is a flowchart illustrating an example technique for generating stimulation or sensing parameters based one monitored signals from a bending sensor associated with the medical lead. For clarity, the techniques of FIG. 15 are described with respect to therapy system 10 (FIG. 1) although other systems are contemplated.

In conjunction with therapy or sensing of brain 20 via medical leads 20, IMD 16 monitors a signal from a bending sensor associated with one or more of medical leads 20 (602), which could be a signal received during implantation of leads 20. IMD 16 and/or programmer 14 evaluates bending of the medical lead based on the monitored signal (604). IMD 16 and/or programmer 14 then selects stimulation or sensing parameters based on the evaluation of the bending of the medical lead (606). For example, IMD 16 and/or programmer 14 may select electrodes for the stimulation and/or sensing functions in order to compensate for mechanical shifts occurring during the positioning of one or more of leads 20 in the brain (e.g., during implantation), or that occur over time within brain 28. The selected electrodes may allow the intended target area within brain 28 to be maintained even when the position of one or more of leads 20 changes. In some examples, IMD 16 may automatically select stimulation or sensing parameters to account for changes in the positions of one or more of leads 20 during implantation and/or over time. In the same or different examples, a user, such as a clinician or patent, may select stimulation or sensing parameters to account for changes in the positions of one or more of leads 20 during implantation and/or over time via programmer 14.

In some examples, bending sensor information may be combined with information from the sensed neural activity on one or more of electrodes 24, 26. Thus the addition of a mechanical sensor (potentially) enables a more sophisticated electro-mechanical closed-loop DBS system than in other examples in which only sensed neural activity on one or more of electrodes 24, 26 is used to select therapy or sensing parameters.

In the same or different examples, a voltage may be applied to piezoelectric elements within a lead or stylet, the piezoelectric elements being part of a bending sensor or separate from a bending sensor, in order to apply a bending force to the lead or stylet. The bending force may be manually selected by a user, such as a clinician, or may be applied in response to a sensed position of the lead. For example, the bending force could be applied to counteract any sensed bending or lead migration during implantation and/or over time in a closed-loop control. The bending force could also be applied on purpose, for example, during insertion to allow steering of the lead during implantation, such as insertion of curved lead to avoid certain brain structures. Such examples may include a flexible stylet including a piezoelectric actuator or another type of microscopic actuator.

In one particular example, sensing and bending by applying a voltage to the piezoelectric elements may occur in a time-interleaved fashion, such as actuation-sensing-actuation-etc. For example, during an implantation procedure, sensing and active bending may be interleaved with small advancements of the lead into patient tissue, such as brain 28. Again the piezoelectric elements may be used for both active bending and sensing or different sensing and actuation mechanisms may be used. In some examples, active bending may occur without use of a bending sensor. In such examples, steering may occur using other techniques, such as sensing with electrodes and/or visual monitoring of lead positioning.

While the techniques described herein are suitable for systems and methods involving DBS therapies, and may be used treat such disorders as Parkinson's disease, Alzheimer's disease, tremor, dystonia, depression, epilepsy, OCD, and other disorders, the techniques are not so limited. One or more such techniques and systems may be applied to treat disorders such as chronic pain disorders, urinary or fecal incontinence, sexual dysfunction, obesity, mood disorders, gastroparesis or diabetes, and may involve other types of stimulation such as spinal cord stimulation, cardiac stimulation, pelvic floor stimulation, sacral nerve stimulation, peripheral nerve stimulation, peripheral nerve field stimulation, gastric stimulation, or any other electrical stimulation therapy. In some cases, the electrical stimulation may be used for muscle stimulation.

In addition, it should be noted that examples of the systems and techniques described herein may not be limited to treatment or monitoring of a human patient. In alternative examples, example systems and techniques may be implemented in non-human patients, e.g., primates, canines, equines, pigs, and felines. These other animals may undergo clinical or research therapies that my benefit from the subject matter of this disclosure.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis. The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. A medical device system comprising:
a medical lead;
a bending sensor; and
a controller configured to sense a bending of the medical lead during implantation of the medical lead in a patient based on the output of the bending sensor.

2. The system of claim 1, further comprising a lead delivery device for implanting the medical lead in the patient, the lead delivery device including the bending sensor.

3. The system of claim 1, wherein the lead delivery device includes at least one of a guide catheter, wherein the lead is configured to be inserted in the guide catheter, or a stylet temporarily insertable into the medical lead, wherein the bending sensor is integrated in and/or on the stylet or guide catheter.

4. The system of claim 3, wherein lead delivery device includes the stylet, wherein the stylet has an outer diameter of less than approximately 1 millimeter.

5. The system of claim 1, wherein the bending sensor is integrated into the medical lead.

6. The system of claim 1, wherein the bending sensor comprises an optical sensor.

7. The system of claim 6, wherein the bending sensor comprises at least one optical fiber.

8. The system of claim 1, wherein the bending sensor comprises a piezoelectric sensor.

9. The system of claim 1, wherein the bending sensor comprises a resistance sensor, which changes its resistance by compression or expansion of resistive material due to the bending of the medical lead.

10. The system of claim 1, wherein the medical lead is a deep brain stimulation medical lead.

11. The system of claim 1, wherein the medical lead has an outer diameter of less than approximately 3.0 millimeters.

12. The system of claim 1, wherein the controller is configured to generate information to guide steering of the medical lead toward a target site based on an evaluation of the bending of the medical lead.

13. The system of claim 1, further comprising an implantable medical device, wherein the implantable medical device is configured to at least one of deliver electrical stimulation to a patient or sense electrical activity of the patient via the medical lead.

14. A method for guiding implantation of a medical lead, the method comprising:
while the medical lead is being inserted into tissue of a patient, monitoring a signal from a bending sensor associated with the medical lead;
evaluating bending of the medical lead during the insertion based on the monitored signal; and
while the medical lead is being inserted into the tissue of the patient, generating information to guide steering of the medical lead toward a target site based on the evaluation of the bending of the medical lead, wherein at least one of the monitoring, evaluating, or generating is performed via a processor.

15. The method of claim 14, further comprising presenting the information to guide steering of the medical lead to a clinician via a user interface.

16. The method of claim 14, wherein the medical lead is inserted into the tissue of the patient via a lead delivery device.

17. The method of claim 14, wherein the lead delivery device includes at least one of a guide catheter, wherein the lead is inserted in the guide catheter to insert the lead into the tissue of the patient, or a stylet temporarily insertable into the medical lead to insert the lead into the tissue of the patient, and wherein the bending sensor is integrated in and/or on the stylet or guide catheter

18. The method of claim 14, wherein the bending sensor is integrated into the medical lead.

19. The method of claim 14, wherein the bending sensor comprises at least one of an optical sensor, a piezoelectric sensor, or a resistance sensor that changes its resistance by compression or expansion of resistive material due to bending.

20. The method of claim 14, wherein the medical lead is a deep brain stimulation medical lead, and the patient tissue includes a brain of the patient.

21. The method of claim 14, further comprising, when the medical lead is implanted adjacent the target site within the patient, at least one of delivering of stimulation therapy via the medical lead or sensing electrical activity of the patient via the medical lead.

22. A system comprising:
a medical lead;
means for monitoring a signal from a bending sensor associated with the medical lead while the medical lead is being inserted into tissue of a patient;
means for evaluating bending of the medical lead during the insertion based on the monitored signal; and
means for generating information, while the medical lead is being inserted into the tissue of the patient, to guide steering of the medical lead toward a target site based on the evaluation of the bending of the medical lead.

23. The system of claim 20, further comprising means for at least one of delivering stimulation therapy to a patient via the medical lead or sensing electrical activity of the patient via the medical lead.
